(19) United States
(12) Patent Application Publication
(10) Pub. No.: US 2024/0374480 A1
(43) Pub. Date: Nov. 14, 2024

(54) Housings, Cameras, And Lights For Use With Gastrointestinal Devices

(71) Applicant: Guofeng Xie, Lutherville Timonium, MD (US)

(72) Inventor: Guofeng Xie, Lutherville Timonium, MD (US)

(21) Appl. No.: 18/766,507

(22) Filed: Jul. 8, 2024

Related U.S. Application Data
(60) Provisional application No. 63/464,744, filed on May 8, 2023.

(51) Int. Cl. A61J 15/00; A61B 1/00; A61B 1/015; A61B 1/05; A61B 1/06; A61M 25/01

(52) U.S. Cl. CPC A61J 15/0084; A61B 1/00009; A61B 1/00045; A61B 1/00103; A61B 1/00114; A61B 1/015; A61B 1/051; A61B 1/0676; A61M 2025/0166

(57) ABSTRACT

Camera devices and methods of use are provided wherein the camera device is attached to a medical device that is placed within a patient's body and the camera device includes a light source, a camera, a housing with adapters, and a cable, wherein the camera captures image data that is used with the cable to assist an operator in the control, placement and use of the medical device and the camera device is capable of being used with several different sized medical devices by being provided with several different sized adapters.

### CROSS REFERENCE TO RELATED APPLICATIONS

This application claims the benefit of U.S. Provisional Application Ser. No. 63/464,744, filed on May 8, 2023, which is hereby incorporated by reference herein.

### FIELD OF THE INVENTION

The invention relates to housings, cameras, and lights for use with gastrointestinal (GI) devices and methods for using such.

### BACKGROUND OF THE INVENTION

Patients often need GI devices for various purposes, such as providing critical nutritional support, GI tract decompression, monitoring GI bleeding, providing diagnosis for patients with difficulty swallowing, etc. Common GI devices include nasogastric tubes (NGT), post-pyloric Dobhoff (Keo) feeding tubes (e.g., small intestinal feeding tubes), rectal tubes, and high-resolution impedance esophageal manometry (HRiM) probes/catheters (in office settings), etc. These devices are often placed blindly and success rates among expert practitioners are approximately 60-90%. Complications can include minor ones such as coiling in the oropharynx, esophagus and stomach, often resulting in failed attempts, and serious and life-threatening ones such as misplacement in the airway causing respiratory compromise, and bleeding or GI tract perforation. The final locations of these devices typically require radiological confirmation, which increases costs and radiation exposure. X-ray imaging also only provides a general location and does not confirm precise locations in most cases.

Currently, the available solutions have significant drawbacks. There is a small intestinal feeding tube with integrated camera (Iris) and associated monitoring devices, but this design is not compatible with existing GI devices, and its high-cost limits adoption by physicians and hospitals. There are no similar devices with NGT, rectal tube or HRiM probes.

For example, typical devices have limited compatibility with various related devices and software used during GI procedures. High costs of typical devices often limit adoption by physicians.

More advantageous solutions with, for example, providing visualization and light, adaptability to different GI devices, increased compatibility with related devices and software, higher adoption by physicians, less complications, better confirmation abilities and/or results, lower costs, and/or lower radiation exposure, are needed and are objects of certain of the embodiments of this invention.

### SUMMARY OF THE INVENTION

Embodiments of this invention comprise a housing (which may comprise an adapter (e.g., sleeves)), camera, and one or more lights for use with GI devices. Preferred embodiments are configured to allow operators to use existing GI devices in both hospital and office settings, such as NGT, Keo tubes, rectal tubes and HRiM probes, to transmit live video and/or other image data to the operator and/or others to guide precise placement of these GI devices, ensure successful placement and avoid serious complications. These embodiments are of relatively low cost, sterile, convenient to use and disposable (e.g., single use). These embodiments may reduce the use of x-ray imaging to verify placement, thereby reducing a patient's exposure to radiation.

In certain preferred embodiments of this invention, the camera is a nano camera configured on a chip or a PCB board. The camera is configured to be placed in a housing having a diameter that is typical of GI devices (e.g., about 2 to about 24 Fr; about 2 to about 8 mm). The camera is configured to provide an electrical signal comprising image data to an operator, including physicians and nurses, using a wired or wireless connection. For example, for a camera using a wired connection the camera is electrically coupled via a cable/wire from the camera in the housing at a distal end of a GI device, alongside or through a lumen in the GI device to a proximal end of the GI device for coupling to a display screen or other processor. The cable can incorporate a metal wire to increase stiffness and assist with placement of these GI devices to overcome coiling in the GI lumen. Also, the camera signal comprising image data can be stored or transmitted to a remote location, such as a processing unit/display, a laptop/desktop computer or a cell or mobile phone for later review by another provider.

In highly preferred embodiments of this invention, camera devices for attachment to medical devices (preferably a GI device) are provided. The medical device has a distal end (toward the patient) and a proximal end (toward the operator) and the medical device is capable of being partially placed inside the body of a patient.

The camera devices comprise (a) a light source (e.g., a plurality of LEDs) connected to a first power source, and (b) a camera for capturing image data connected to (i) the first power source (e.g., a battery) and/or a second power source (e.g., an outlet in an operating room, a laptop computer), and (ii) an interface with a computer processor.

The camera devices also comprise (c) a housing containing the light source and the camera. The housing is attachable to the distal end of the medical device using an adapter. In some embodiments, clips are used to attach the housing to the medical device. In some embodiments, the camera device housing is provided with multiple sized adapters that each fit a different medical device, so that one camera device can be used with different medical devices using the correct fitting adapter for each.

The camera devices also comprise (d) a cable attached to the camera device on one end, the cable extending to the proximal end of the medical device, wherein the cable assists with the placement of the medical device inside the body of the patient. The cable may comprise a stiffening wire in some embodiments to help with this placement.

The camera captures image data that is enhanced by the light source and which is transmitted through the interface to the computer processor.

In certain preferred embodiments, the medical device is a gastrointestinal device for examination, diagnosis, and/or treatment of the gastrointestinal system of the patient. In the most preferred embodiments, the medical device is selected from the group consisting of a nasogastric tube, a small intestine Keo feeding tube, a rectal tube, and an esophageal manometry probe.

In certain preferred embodiments, the housing comprises an adapter that attaches to the distal end of the medical device. In the most preferred embodiments, the camera device is provided with a plurality of different sized adapters that can each be separately used to attach to the distal end of different sized medical devices. In some of these embodiments, the plurality of different sized adapters range in size from about 2 mm to about 8 mm in diameter. In some embodiments of adapters, the adapters comprise one or more passages to permit the passage of air and/or fluids through the housing.

In certain preferred embodiments, the cable connects the camera to the first power source and/or the second power source, and the cable transmits the image data through the interface to the computer processor. In some embodiments, the cable comprises a passage for air for insufflation and/or liquid for cleaning the camera lens. In some embodiments, the cable comprises wire (e.g., stiffening wire) to assist with placement of the medical device.

In certain preferred embodiments, the computer processor is in a display device, a desktop computing device, a laptop computing device, a tablet computing device, or a mobile phone device, and the computer processor provides direct live viewing and recording of the image data including of blood and any active gastrointestinal bleeding during placement of the medical device.

In the most preferred embodiments, the camera device is a single use device and it is disposed of after one use.

In another highly preferred embodiment of this invention, methods for an operator conducting gastrointestinal medical procedures with medical devices are provided. Each medical device has a distal end and a proximal end, and the medical device is capable of being partially placed inside the body of a patient, The medical device is preferably a nasogastric tube, a small intestine Keo feeding tube, a rectal tube, or an esophageal manometry probe.

These methods comprise: (a) attaching a camera device to the medical device. The camera device comprises (i) a light source connected to a first power source; (ii) a camera for capturing image data connected to (A) the first power source and/or a second power source, and (B) an interface with a computer processor; (iii) a housing containing the light source and the camera, the housing comprising an adapter, wherein the housing is attachable to the distal end of the medical device using the adapter; and (iv) a cable attached to the camera device on one end, the cable extending to the proximal end of the medical device, wherein the cable assists with the placement of the medical device inside the body of the patient.

The methods also comprise (b) capturing image data with the camera device; (c) transmitting the image data to the computer processor; and (d) displaying the image data for the operator to see (and use the image data to conduct the procedure). The use of these camera devices and methods allows for several advantages, including more accurate placement of the medical devices and timely and sensitive detection of blood/active bleeding in the GI tract without the use of endoscopes, which are often not available and which may be expensive and relatively risky to use.

Certain embodiments of this invention are configured to provide one or more advantages, uses and/or features, including but not limited to: (a) increase visualization and/or light, (b) increased accuracy and precision of placement of GI devices during procedures, (c) reduced cost of GI device placement, (d) a lower cost for a camera and/or light, (e) being disposable, (f) reduced use of x-ray imaging to confirm placement of GI devices, therefore having the potential capability to reduce a patient's exposure to radiation, (g) improved success and outcomes with visualization, diagnosis, and/or treatment, (h) application and adaptability to one or more GI devices, and preferably a variety of GI devices, and/or (i) allowing initial assessment of GI bleeding via direct visualization. Further advantages, uses and/or features will be apparent to a person of skill in the art applying embodiments of the invention, and the foregoing list is not limiting.

Applications for the preferred embodiments of this invention include their use with GI devices and methods of their use, in the examination, visualization, placement/localizing, reporting, diagnosis, and treatment of disease and patient anatomy, and other uses where visualization may be helpful. The person of ordinary skill in the art understands how these applications can be done.

Advantages of the embodiments of this invention are described and apparent throughout this specification. For example, certain embodiments provide increased compatibility with related devices and software, higher adoption by physicians, less complications, better confirmation abilities and/or results, lower costs, and/or lower radiation exposure, among other advantages. Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

FIG. 1 illustrates an exemplary embodiment of a camera of this invention shown by two different perspective views of the camera.

FIG. 2 illustrates a progression of the connection of components of an embodiment of this invention, including (A) perspective views of a camera and lights to be inserted in a distal portion of a housing, (B) a perspective view of the housing and its adapter or adaptor, (C) perspective views of cables and connectors for a camera device, and (D) a perspective view of a PCB board to be connected to a camera device.

FIG. 3 illustrates three perspective views of an embodiment of a camera device of this invention, including (A) an outer perspective view of a camera device with an adapter of a housing unscrewed from the camera device, (B) an inner perspective view of certain components of the camera device, and (C) an outer perspective view of the camera device.

### DETAILED DESCRIPTION OF THE INVENTION

The most preferred embodiments of this invention comprise a camera, and one or more lights, each of which are provided together and are within and/or attached to a housing, which can be attached to a GI device. These embodiments further comprise a cable that assists with the placement of the GI device.

In certain preferred embodiment of this invention, the camera (e.g., a nano camera) is configured on a chip or printed circuit board (PCB board).

The cameras in these preferred embodiments are also configured to be placed in a housing having a diameter that is of a size and shape typical of GI devices. These cameras are configured to provide an electrical signal comprising image data using a wired or wireless connection. For example, in some embodiments with a camera using a wired connection, the camera is electrically coupled via a wire from the camera in the housing at a distal end of a GI device, through a lumen in the GI device to a proximal end of the GI device for coupling to a screen (e.g., monitor) and/or processor. For a wireless connection, a wireless signal is provided by the camera to a screen (e.g., monitor) and/or processor through a wireless connection.

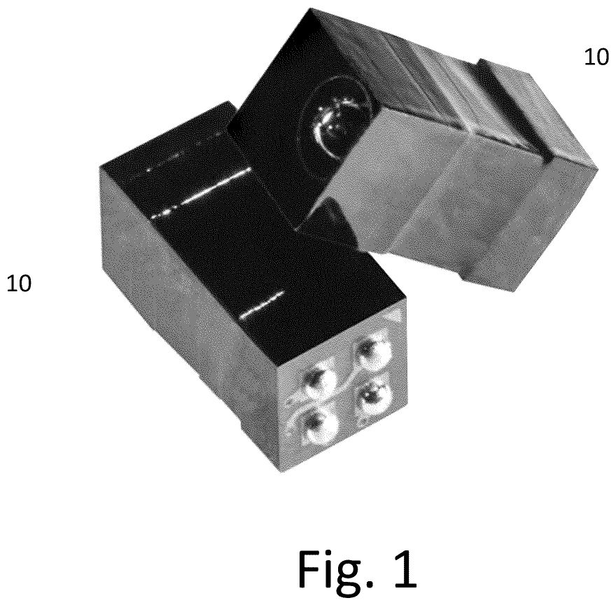

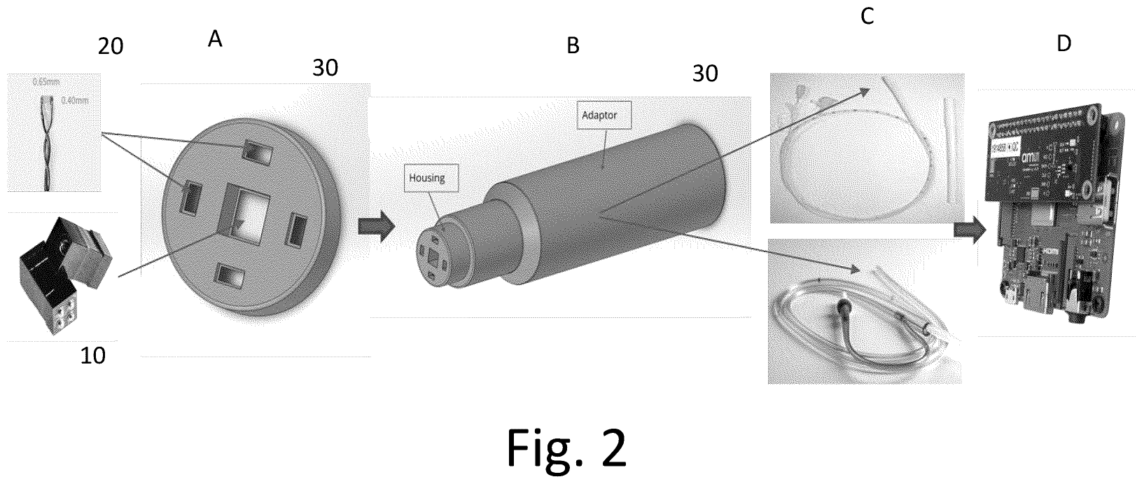

FIG. 1 provides an example of a camera 10 of this invention shown in two different perspective views. FIG. 2A also shows this camera 10.

In some particularly preferred embodiments, the camera signal comprising image data can be stored or transmitted to a remote location, such as for later review and/or an offsite physician.

In certain preferred embodiments of this invention, housings are provided that each house (e.g., contain, attach, connect to) a camera and at least one light to protect the cameras and lights from environmental harm and to provide a sterile environment for using the device. The housings may comprise an adapter that attaches to a GI device.

In certain of these embodiments, the housings (e.g., and their adapter (or adaptor) component (e.g., sleeve)) are configured to fit with (e.g., onto, into) an existing GI device, such as those noted above (e.g., NGT, Keo tubes, rectal tubes, HRiM probes, etc.). In some of these embodiments, the housings have a diameter that is approximately the same size and cross section as the GI device(s) of interest for the particular application. For example, the diameter of the housing in some embodiments can be between 6 to 18 French or FR and have a generally circular or elliptical cross-section. However, smaller and larger housings are feasible for different procedure and patient needs. In some embodiments, the housings include radiopaque materials for viewing using typical imaging techniques.

In certain preferred embodiments of this invention, the housings can be attached to a GI device using typical and low-cost methods, well known to a person of skill in the art. For example, the housings in some embodiments are mechanically attached to a GI device by a fictional fit, wherein the housing fits tightly over the distal end of a GI device such that fiction retains it in place while it is in use in a patient. In some embodiments, the housings can threadably attach to GI devices by an adapter component of the housing. In some embodiments, the housings attach to GI devices using an adhesive. In some embodiments, the housings can include a lock that is configured to fix the housing onto a GI device such that it is less likely to unintentionally fall off. Furthermore, the lock in certain of these embodiments may be configured to maintain the relative position (e.g., axial and/or radial position) of the housing with respect to distal and proximal ends of the GI device.

FIG. 2 provides an illustration of the progression of exemplary components of this invention, including a housing 30 of this invention and how the camera 10 and lights 20 fit into the housing 30 in FIG. 2A in this particular embodiment. FIG. 2B shows an adapter (or adaptor) of a housing 30. FIG. 2C shows other components to which a camera device of this invention may attach. FIG. 2D shows a connection of a camera device to an embodiment of a computer processor.

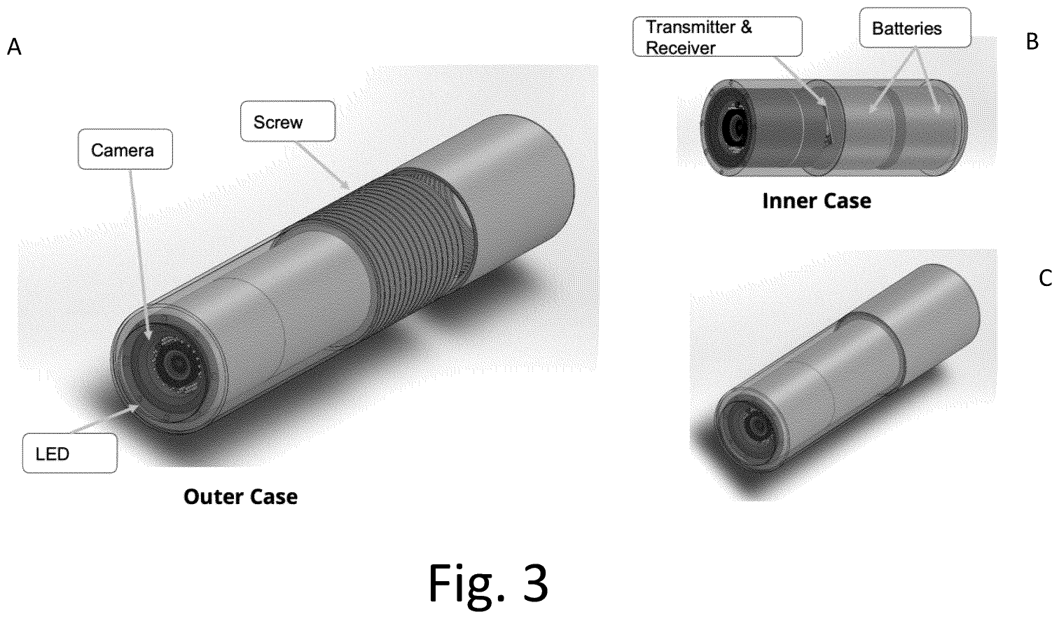

FIG. 3 provides an illustration of a different embodiment than FIG. 2. An LED light is shown, as well as a camera, in FIG. 3A. An adaptor is shown screwed off of a housing in FIG. 3A and screwed on to a housing in FIG. 3C. An internal view of components of a camera device embodiment is shown in FIG. 3B, including a wireless embodiment with a transmitter and receiver and batteries.

In certain embodiments of this invention, the lights are configured to illuminate a cavity or lumen in patients to facilitate placement of the GI devices. For example, the lights can provide illumination for the camera to provide a signal comprising image data that indicates a position of the proximal end of the GI device, wherein an operator or controller can recognize the position of the proximal end by identifying landmarks or indicators of a patient's anatomy when viewed on a screen (e.g., monitor, other display) and/or the data is processed to provide a location. Furthermore, each of the lights may comprise a plurality of components providing light.

In certain embodiments of this invention, each of the lights is comprised of a plurality of LED light components. In some of these embodiments, the plurality of LED light components are at least a pair of LED light components configured to substantially and uniformly illuminate a desired cavity or lumen. For example, the plurality of LED light components can be oriented and distributed on the housing to substantially and uniformly illuminate a desired cavity or lumen and be configured for specific cavities or lumens for specific procedures.

In certain embodiments of this invention, the lights are each powered by batteries that are preferably within the housing. In some of these embodiments, this configuration is used with a wireless signal from the camera (e.g., as described above).

In certain embodiments of this invention, the lights are each powered by a wired connection to an external power source, such as to a power source in an operating room, a power source on a GI device, and/or a power source in a desktop/laptop computer or cell or mobile phone.

In some embodiments of this invention, the lights are each powered by batteries with a backup or alternative wired connection to an external power source.

In certain embodiments of this invention, the cameras are configured to transmit signals comprising image data relating to a cavity or lumen.

In certain embodiments of this invention, the cameras are each powered by batteries that are preferably within the housing. In some embodiments, the cameras transmit signals comprising image data over a wireless signal (e.g., as described above) and a transmitter and receiver are used.

In certain embodiments of this invention, the cameras are each powered by a wired connection to an external power source, such as to a power source in an operating room, a power source on a GI device, and/or a power source in a desktop/laptop computer or cell or mobile phone.

The subject matter of this disclosure is now described with reference to the following example. This example is provided for the purpose of illustration only, and the subject matter is not limited to this example, but rather encompasses all variations which are evident as a result of the teaching provided herein.

### Example 1

In this particularly preferred embodiment, a disposable camera device is provided that comprises a housing, a camera, a light source, and a cable. The housing comprises a set of various adapters in the form of different sized sleeves that allows the disposable camera device to fit a variety of GI devices, including NGTs, small intestinal feeding tubes, rectal tubes, and HRiM probes/catheters. The camera can be connected to a processing unit/display, laptop/desktop computer, or cell or mobile phone to provide power and allow direct viewing of the procedure with the GI device. The user or operator is assisted with the placement of the device with the imaging data, which increases the chance of successful completion of the procedure and the prevention of potential life-threatening complications. The user is also assisted with the placement of the device by the cable, which can include a metal stiffening wire, to assist in the movement of the GI device to the correct location and to avoid coiling in the GI lumen. In addition, the use of these camera devices allows timely and sensitive detection of blood/active bleeding in the GI tract without the use of endoscopes, which are often not available and which may be expensive and relatively risky to use.

### OTHER EMBODIMENTS

Although the present invention has been described with reference to teaching, examples and preferred embodiments, one skilled in the art can easily ascertain its essential characteristics, and without departing from the spirit and scope thereof can make various changes and modifications of the invention to adapt it to various usages and conditions. Those skilled in the art will recognize or be able to ascertain using no more than routine experimentation, many equivalents to the specific embodiments of the invention described herein. Such equivalents are encompassed by the scope of the present invention.

What is claimed is:

1. A camera device for attachment to a medical device, the medical device having a distal end and a proximal end, the medical device being capable of being partially placed inside the body of a patient, the camera device comprising:
a. a light source connected to a first power source;
b. a camera for capturing image data connected to (i) the first power source and/or a second power source, and (ii) an interface with a computer processor;
c. a housing containing the light source and the camera, the housing comprising a plurality of different sized adapters, wherein the housing is attachable to the distal end of the medical device using one of the adapters;
d. a cable attached to the camera device on one end, the cable extending to the proximal end of the medical device, wherein the cable increases stiffness to avoid coiling of the medical device and assists with the placement and use of the medical device inside the body of the patient; and
e. wherein the camera captures image data that is enhanced by the light source and which image data is transmitted through the interface to the computer processor which is then used to display the image data to provide information concerning the patient and the use of the medical device.

2. The camera device of claim 1, wherein the medical device is a gastrointestinal device for examination, diagnosis, and/or treatment of the gastrointestinal system of the patient.

3. The camera device of claim 1, wherein the medical device is selected from the group consisting of a nasogastric tube, a small intestine Keo feeding tube, a rectal tube, and an esophageal manometry probe.

4. The camera device of claim 1, wherein the adapter that attaches to the distal end of a nasogastric tube, a small intestine Keo feeding tube, a rectal tube, or an esophageal manometry probe.

5. The camera device of claim 1, wherein the adapter comprises one or more passages to permit the passage of air and/or fluids through the housing.

6. The camera device of claim 1, wherein the cable connects the camera to the first power source and/or the second power source, and the cable transmits the image data through the interface to the computer processor.

7. The camera device of claim 6, wherein the cable comprises a passage for air for insufflation and/or liquid for cleaning the camera lens.

8. The camera device of claim 7, wherein the cable comprises wire to assist with placement of the medical device.

9. The camera device of claim 1, further comprising one or more clips to attach the housing to the medical device.

10. The camera device of claim 1, wherein the computer processor is in a display device, a desktop computing device, a laptop computing device, a tablet computing device, or a mobile phone device, and the computer processor provides direct live viewing and recording of the image data including of blood and any active gastrointestinal bleeding during placement of the medical device.

11. The camera device of claim 1, wherein the camera device is a single use device and it is disposed of after one use.

12. A method for an operator conducting a gastrointestinal medical procedure with a medical device, the medical device having a distal end and a proximal end, the medical device being capable of being partially placed inside the body of a patient, the medical device being selected from the group consisting of a nasogastric tube, a small intestine Keo feeding tube, a rectal tube, and an esophageal manometry probe, the method comprising:
a. attaching a camera device to the medical device, the camera device comprising (i) a light source connected to a first power source; (ii) a camera for capturing image data connected to (A) the first power source and/or a second power source, and (B) an interface with a computer processor; (iii) a housing containing the light source and the camera, the housing comprising an adapter, wherein the housing is attachable to the distal end of the medical device using the adapter; and (iv) a cable attached to the camera device on one end, the cable extending to the proximal end of the medical device, wherein the cable assists with the placement of the medical device inside the body of the patient;
b. capturing image data with the camera device;
c. transmitting the image data to the computer processor; and
d. displaying the image data for the operator to see.

13. A camera device for attachment to one of a plurality of different sized gastrointestinal medical devices, each of the medical devices having a distal end and a proximal end, the medical devices being capable of being partially placed inside the body of a patient, the camera device comprising:
a. a light source connected to a first power source;
b. a camera for capturing image data connected to (i) the first power source and/or a second power source, and (ii) an interface with a computer processor;
c. a housing containing the light source and the camera, the housing comprising a plurality of different adapters each of which is sized to fit with one of the plurality of medical devices, wherein the housing is attachable to the distal end of one of the medical devices;
d. a cable attached to the camera device on one end, the cable extending to the proximal end of one of the medical devices, wherein the cable assists with the placement of the medical device inside the body of the patient; and
e. wherein the camera captures image data that is enhanced by the light source and which image data is transmitted through the interface to the computer processor which is then used to display the image data to provide information concerning the patient and the use of the medical device, such information including the detection of bleeding in the patient.
